SEQUENCE LISTING (3 sequences)

SEQ ID NO: 1 - protein, 75 aa, Unknown
  Envelope Protein Listed in GENBANK as YP_009724392.1 The sequence was the translated from NC_045512.2 DNA found in Genbank for Novel Coronavirus for Figure 2b calculation
MYSFVSEETGTLIVNSVLLFLAFVVFLLVTLAILTALRLCAYCCNIVRVSLVKPSFYVYSRVKDLDSSRVPDLLV

SEQ ID NO: 2 - protein, 1273 aa, Unknown
  Spike Protein Listed in GENBANK as YP_009724390.1 . The sequence was the translated from NC_045512.2 DNA found in Genbank for Novel Coronavirus for Figure 2a calculation
MFVFLVLLPLVSSECVNLTTRTELPPAYTNSFTRGVYYPDKVFRSSVLHSTEDLFLPFFSNVTWFHAIHVSGTNGTKRFDNPVLPFNDGVYFASTEKSNIIRGWIFGTTLDSKTESLLIVNNATNVVIKVCEFEFCNDPFLGVYYHKNNKSWMESEFRVYSSANNCTFEYVSEPFLMDLEGKEGNFKNLREFVFKNIDGYFKIYSKHTPINLVRDLPEGFSALEPLVDLPIGINITRFETLLALHRSYLTPGDSSSGWTAGAAAYYVGYLEPRTFLLKYNENGTITDAVDCALDPLSETKCTLKSFTVEKGIYETSNFRVEPTESIVRFPNITNLCPFGEVFNATRFASVYAWNRKRISNCVADYSVLYNSASFSTFKCYGVSPTKLNDLCFTNVYADSFVIRGDEVREIAPGETGKIADYNYKLPDDFTGCVIAWNSNNLDSKVGGNYNYLYRLFRKSNLKPFERDISTEIYEAGSTPCNGVEGFNCYFPLESYGFEPTNGVGYEPYRVVVLSFELLHAPATVCGPKKSTNLVKNKCVNFNFNGLTGTGVLTESNKKFLPFEEFGRDIADTTDAVRDPETLEILDITPCSFGGVSVITPGTNTSNEVAVLYEDVNCTEVPVAIHADELTPTWRVYSTGSNVFETRAGCLIGAEHVNNSYECDIPIGAGICASYETETNSPRRARSVASESIIAYTMSLGAENSVAYSNNSIAIPTNFTISVTTEILPVSMTKTSVDCTMYICGDSTECSNLLLEYGSFCTELNRALTGIAVEEDKNTEEVFAEVKEIYKTPPIKDFGGFNFSEILPDPSKPSKRSFIEDLLFNKVTLADAGFIKEYGDCLGDIAARDLICAEKFNGLTVLPPLLTDEMIAEYTSALLAGTITSGWTFGAGAALEIPFAMEMAYRFNGIGVTENVLYENEKLIANEFNSAIGKIEDSLSSTASALGKLEDVVNENAEALNTLVKELSSNFGAISSVLNDILSRLDKVEAEVEIDRLITGRLESLETYVTEELIRAAEIRASANLAATKMSECVLGESKRVDFCGKGYHLMSFPESAPHGVVFLHVTYVPAEEKNFTTAPAICHDGKAHFPREGVFVSNGTHWFVTERNFYEPEIITTDNTFVSGNCDVVIGIVNNTVYDPLEPELDSFKEELDKYFKNHTSPDVDLGDISGINASVVNIEKEIDRLNEVAKNLNESLIDLEELGKYEEYIKWPWYIWLGFIAGLIAIVMVTIMLCCMTSCCSCLKGCCSCGSCCKFDEDDSEPVLKGVKLHYT

SEQ ID NO: 3 - protein, 1197 aa, Unknown
  Surface Glycoprotein Mutant missing Isoleucines for example calculation of Figure 2c Fictitious derrived from YP_009724390.1 . The sequence was the translated from NC_045512.2 DNA found in Genbank for Novel Coronavirus. The Isoluecenes are removed to show mutation impact on probability folding calculation shown in Figure 2c for example discussion purposes only
MFVFLVLLPLVSSECVNLTTRTELPPAYTNSFTRGVYYPDKVFRSSVLHSTEDLFLPFFSNVTWFHAHVSGTNGTKRFDNPVLPFNDGVYFASTEKSNRGWFGTTLDSKTESLLVNNATNVVKVCEFEFCNDPFLGVYYHKNNKSWMESEFRVYSSANNCTFEYVSEPFLMDLEGKEGNFKNLREFVFKNDGYFKYSKHTPNLVRDLPEGFSALEPLVDLPGNTRFETLLALHRSYLTPGDSSSGWTAGAAAYYVGYLEPRTFLLKYNENGTTDAVDCALDPLSETKCTLKSFTVEKGYETSNFRVEPTESVRFPNTNLCPFGEVFNATRFASVYAWNRKRSNCVADYSVLYNSASFSTFKCYGVSPTKLNDLCFTNVYADSFVRGDEVREAPGETGKADYNYKLPDDFTGCVAWNSNNLDSKVGGNYNYLYRLFRKSNLKPFERDSTEYEAGSTPCNGVEGFNCYFPLESYGFEPTNGVGYEPYRVVVLSFELLHAPATVCGPKKSTNLVKNKCVNFNFNGLTGTGVLTESNKKFLPFEEFGRDADTTDAVRDPETLELDTPCSFGGVSVTPGTNTSNEVAVLYEDVNCTEVPVAHADELTPTWRVYSTGSNVFETRAGCLGAEHVNNSYECDPGAGCASYETETNSPRRARSVASESAYTMSLGAENSVAYSNNSAPTNFTSVTTELPVSMTKTSVDCTMYCGDSTECSNLLLEYGSFCTELNRALTGAVEEDKNTEEVFAEVKEYKTPPKDFGGFNFSELPDPSKPSKRSFEDLLFNKVTLADAGFKEYGDCLGDAARDLCAEKFNGLTVLPPLLTDEMAEYTSALLAGTTSGWTFGAGAALEPFAMEMAYRFNGGVTENVLYENEKLANEFNSAGKEDSLSSTASALGKLEDVVNENAEALNTLVKELSSNFGASSVLNDLSRLDKVEAEVEDRLTGRLESLETYVTEELRAAERASANLAATKMSECVLGESKRVDFCGKGYHLMSFPESAPHGVVFLHVTYVPAEEKNFTTAPACHDGKAHFPREGVFVSNGTHWFVTERNFYEPETTDNTFVSGNCDVVGVNNTVYDPLEPELDSFKEELDKYFKNHTSPDVDLGDSGNASVVNEKEDRLNEVAKNLNESLDLEELGKYEEYKWPWYWLGFAGLAVMVTMLCCMTSCCSCLKGCCSCGSCCKFDEDDSEPVLKGVKLHYT

(19) United States
(12) Patent Application Publication
(10) Pub. No.: US 2022/0370398 A1
(43) Pub. Date: Nov. 24, 2022

(54) METHOD TO DESTABALIZE CYSTEINE EXPOSED INFECTIOUS INFORMATION VECTORS

(71) Applicant: John Scott Perez, Tampa, FL (US)

(72) Inventor: John Scott Perez, Tampa, FL (US)

(21) Appl. No.: 17/210,366

(22) Filed: Mar. 23, 2021

(51) Int. Cl. A61K 31/255; A61K 9/00; A61K 47/10; C12P 11/00; A61K 31/198; A61K 33/04; A61K 33/00; A61P 31/00; A61K 31/05; A61K 31/352; A61K 31/4178

(52) U.S. Cl. CPC A61K 31/255; A61K 9/0078; A61K 47/10; C12P 11/00; A61K 31/198; A61K 33/04; A61K 33/00; A61P 31/00; A61K 31/05; A61K 31/352; A61K 31/4178

(57) ABSTRACT

A vapor viable formula for sanitizing the lungs in order to shield them from a digitally encoded pathogen. The digital code translates into 2 and 3 dimensional proteins capable of self-replicating in the host. Agent(s) can traverse the semipermeable membranes of the host cells in order to specifically target the translated proteins in a 2-dimensional state before they fold into a functional 3-dimensional molecule. The agent(s) must be able to disable the 3-dimensional functional analog state of a pathogen component. 2-Propene-1-sulfinothioic acid S-2-propenyl or an R group derivative thereof is formulated so that it can be put into vapor form and inhaled into the sinuses, throat, bronchi, or lungs with maximum surface bioavailability. Selenium or Selenocysteine exploits of the digital pathogen code are leveraged using vapor formulas. Vapor delivery allows maximum analog or digital exploitation of the pathogen.

### CROSS REFERENCE TO RELATED APPLICATION

This application claims priority under 35 U.S.C. 119€ to U.S. provisional patent application No. 62/993,481 entitled “METHOD TO DESTABALIZE CYSTEINE EXPOSED INFECTIOUS INFORMATION VECTORS,” which was filed Mar. 23, 2020, and which is incorporated herein by reference in its entirety.

### SEQUENCE LISTING INCORORATION BY REFERENCE

Information contained in three sequence listings are submitted in an ASCII text file; the information contained in the three sequence listings are herein incorporated by reference in their entireties. The submitted sequence listings file is:

210818_JSP_003_NCov_Spike_Envelop_Surface_protein_sequences_v10, created on Aug. 18, 2021, and having a file size of 6KB.

### OTHER PUBLICATIONS

1—Importance of Conserved Cysteine Residues in the Coronavirus Envelope Protein, Journal of Virology. Lisa A. Lopez, Ambere J. Riffle, Steven L. Pike, Douglas Gardner, Brenda G. Hogue. DOI: 10.1128/JVI.01914-0
2—SARS-coronavirus spike S2 domain flanked by Cysteine residues C822 and C833 is important for activation of membrane fusion. Virology. Madu IG¹, Belouzard S, Whittaker G R. 2009 Oct. 25; 393(2):265-71. doi:10.1016/j.virol.2009.07.038. Epub 2009 Aug. 29.

### 3—Leishenshan (Thunder God Mountain) Hospital in Wuhan, Peng Zhiyong

4—Diallylthiosulfinate (Allicin), a Volatile Antimicrobial from Garlic (Allium sativum), Kills Human Lung Pathogenic Bacteria, Including MDR Strains, as a Vapor. Molecules Jana Reiter, Natalja Levina , Mark van der Linden , Martin Gruhlke , Christian Martin and Alan J. Slusarenko 2017 Oct. 12; 22(10):1711
5—The human allicin-proteome: S-thioallylation of proteins by the garlic defence substance allicin and its biological effects. Martin C. H. Gruhlkea,*,1, Haike Antelmannb,1, Jorg Bernhardtc, Veronika Kloubertd, Lothar Rinkd, Alan J. Slusarenkoa
6—Nutraceuticals Efficacy, Safety and Toxicity 2016, Pages 555-568 Chapter 41-Organosulfur Compounds as Nutraceuticals Nikolay Goncharov Alexander, N. Orekhov, Natalia Voitenko, Anton Ukolov, Richard Jenkins, Pavel Avdonin. Available online 19 Feb. 2016.
7—Association between regional Selenium status and reported outcome of COVID-19 cases in China Jinsong Zhang, Ethan Will Taylor, Kate Bennett, Ramy Saad, Margaret P Rayman The American Journal of Clinical Nutrition, Volume 111, Issue 6, June 2020, Pages 1297-1299, Published: 28 Apr. 2020
8—Cellular Selenoprotein mRNA Tethering via Antisense Interactions with Ebola and HIV-1 mRNAs May Impact Host Selenium Biochemistry. Ethan Will Taylor, Jan A. Ruzicka, Lakmini Premadasa, Lijun Zhao. Current Topics in Medicinal Chemistry: Volume 16, Issue 13, 2016
9—Direct verbal meetings with Dr. Christopher Kevil, Louie K. Scott, and Anthony W. Orr regarding clinical research June 2020.
10—Angiotensin II Mediates Angiotensin Converting Enzyme Type 2 Internalization and Degradation Through an Angiotensin II Type I Receptor-Dependent Mechanism Matthew R. Deshotels*, Huijing Xia*, Srinivas Sriramula, Eric Lazartigues, and Catalin M. Filipeanu Hypertension: Volume 64, Issue 6, December 2014, Pages 1368-1375
11—The enthalpy change in protein folding and binding: refinement of parameters for structure-based calculations. V J Hilser, J Gomez, E Freire Proteins: 1996 October; 26(2):123-33
12—On the origin of the enthalpy and entropy convergence temperatures in protein folding. LITIAN FU AND ERNESTO FREIRE. Proc. Natl. Acad. Sci. USA Vol. 89, pp. 9335-9338, October 1992
13—SARS-CoV2 induced respiratory distress: Can cannabinoids be added to anti-viral therapies to reduce lung inflammation? Siddappa N. Byrareddy and Mahesh Mohan. Brain Behavior Immun. 2020 July; 87: 120-121. Apr. 28, 2020.
14—In search of preventive strategies: novel high-CBD Cannabis sativa extracts modulate ACE2 expression in COVID-19 gateway tissues Bo Wang, Anna Kovalchuk, Dongping Li, Rocio Rodriguez-Juarez, Yaroslav Ilnytskyy, Igor Kovalchuk, Olga Kovalchuk. Aging November, 2020. Volume 22; 12(22):22425-22444.
15—Identification of Coronavirus Isolated from a Patient in Korea with COVID-19 Jeong-Min Kim, Yoon-Seok Chung, Hye Jun Jo, Nam-Joo Lee, Mi Seon Kim, Sang Hee Woo, Sehee Park, Jee Woong Kim, Heui Man Kim, and Myung-Guk Han Osong Public Health Res Perspect. 2020 February; 11(1):3-7. doi:10.24171/j.phrp.2020.11.1.02 v.11(1); 2020 February PMC7045880

### FIELD

The field relates generally to the sanitization of the sinuses and lungs by treatment with a vapor formula that can bring therapeutics to the lungs immediately with high bioavailability.

### BACKGROUND

Respiratory antigen treatments using novel molecules that are unstable when heated has been for the most part unavailable to the general public because the technology to deliver these molecules has been unavailable to the general public. Vapor based heating technology from the tobacco and cannabis industries have recently emerged as those industries have been allowed to evolve away from smoking of tobacco and cannabis products. The U.S. Government allows the consumption of these specific products by consumers. The emergence of different methods of gentle vaporization have become commercially available as a result of these markets. None of the market applications have been outside the scope of the tobacco or cannabis markets. Many formulas exist for the solubilization of tobacco and cannabis plant extract technologies but none exist for treating digitally encoded antigens. The emergence of digitally encoded antigens has resulted in respiratory diseases for mammalian organisms such as humans. This emergence illustrated to the populations of the Earth a need to treat said respiratory antigens with a novel method in order to provide sanitization, shielding, disablement of the antigen function for the purpose of providing immunity to the host organism. The use of vapor technology to provide a safe molecular agent directly to the surface of the sinuses or lungs with optimal bioavailability is required in order to achieve such sanitization and immunity goals. The ability to identify and formulate agents capable of performing such function is now in demand for human organisms. Bacteria, viruses, mold, fungi, etc. are all example of digitally encoded antigenic vectors that leverage Deoxy Ribonucleic Acid or Ribonucleic Acid as digital languages.

### SUMMARY

On Feb. 2, 2020 the GENBANK DNA, RNA, and protein sequences of novel coronavirus-19 NC_045512.2 (N-CoV) 100 were reviewed by CannDetect LLC for the purpose of generating the probabilities of the critical proteins of novel N-CoV as well as the homology with of Severe Acute Respiratory Syndrome (SARS). It was clear that the SARS coronavirus had 80% homology 101 with N-CoV virus from the GENBANK BLAST analysis. Sequence homology of SARS-CoV-2 with SARS-CoV, and MERS-CoV was 77.5% and 50%, respectively per widely published in the Scientific literature (15).

The probabilities of these N-CoV viral proteins were then calculated using the 2-dimensional (2D) permutation of sequences formula for the Spike Protein 102 , the Envelope Protein 103 , and the Surface Glycoprotein 104 as shown in FIGS. 2a-c calculations.

The art of 3D protein folding in Biochemistry is a very complicated Physical Chemistry Art that is traditionally validated by both Biochemical Molecular Biology assays in vitro, in vivo, and ultimately through X-ray Crystallography structural validation. The 2D structure can reveal many things about the Genomic DNA and protein homology of a virus or organism. The exercise revealed that the probabilities of getting a functional 3-Dimensional antigen functional viral assembled vector structure at thermostable equilibrium with ΔG=0 with the proper fold were extremely low (FIG. 3).

It is known that the N-CoV can mutate like SARS and maintain functionality. This means that the real-life probabilities of a 2D sequence folding to a functional structure are actually a little less than our sequence calculations (FIGS. 2a-c). Currently those numbers are unknown.

That would simply mean that the probabilities would move from 1 in 10^{x} to something like n values in 10^{x} combination, and that there would be more 2D sequences that could fold into functional 3D proteins in N-CoV after a normal mutation caused during replication. This is in fact observed in nature. However, the glaring high probabilities 102, 103, 104 relative to the permutation of sequences for the amino acid combinations reveals astronomically high probabilities of getting a proper 3D protein fold. Currently, 1 in 10e+56 is considered an absurd probability in Physics. This was an indication that there had to be highly conserved functional sequences maintained in this class of virus that contribute to a very specific viral function. It was already known that SARS had an 80% GENBANK BLAST DNA & RNA homology with N-CoV 101. The next exercise was to look for publications involving the SARS genome and conserved regions. Typically this is a very ominous task, but here we lucked out and found two publications on Feb. 2, 2020 that revealed the conserved amino acids in the SARS family genome as published by Lopez et al, and Madu et al (1)(2) that exist in all coronaviruses 105. The first protein sequence was found in the N-CoV Envelope FIG. 4. This was performed by taking the Homology map of the SARS retrovirus and manually aligning the N-CoV protein sequence for the Envelope. Here a manual sequence alignment was performed by CannDetect, LLC to the publication by Madu et al (2) 106.

This revelation highlighted in yellow which was first observed by the team who published the Virology article Madu et al (2) had already found conserved Cysteine residues in the SARS viral family. FIG. 4 showed that the same N-CoV Cysteine residues in red labeling manually lined up in the exact amino acid alignment with N-CoV per the yellow highlight in FIG. 4 105. Similarly the same manual alignment exercise was performed on the second publication in the Journal of Virology by Lopez et al (1). This was a very big finding that revealed a conserved SARS cleavage point required by the N-CoV that was not only maintained but required for cellular invasion by the virus. Ironically these were conserved Cysteines again 107. The amino acid sequence of the N-CoV 108 was then manually aligned with the amino acid sequence of the SARS genome to reveal the Cysteines in N-CoV were also conserved in FIG. 5

Mutation experiments in mice showed that removal of a single Cysteine required in the 2D sequence for a critical Cysteine bridge motif in the spike or envelope rendered the SARS virus as no longer functional and unable to replicate (1)(2). These were critical validation of the sequences that the N-CoV had exposed Cysteine amino acids 107 that were viable drug targets, and it was no longer necessary to solve the X-ray crystal structure of the viral proteins at this point. SARS reference to 3D protein known structures could be used as a reference due to protein sequence homology. Now the focus moved to a means of using known Organic Chemistry (6, Gruhlke et al) by to negate access to the exposed Cysteines to activation by cell surface proteins. The first method is to use 2-Propene-1-sulfinothioic acid S-2-propenyl ester (2PSP) 109 a molecule that is known to bind directly 112 to Cysteine amino acids 111 in proteins when exposed. FIG. 6 is the molecular structure of 2PSP 109.

This molecule will break in half producing two sulfonated propenes FIG. 6a when reacting with Cysteine in vivo 111. Those 2 sulfonated propenes will then bind Cysteine FIG. 6b. The reaction is shown in FIG. 6c.

The reactions shown in 6c 112 or 6e 113 block the flanking Cysteines 114, 115 shown in FIGS. 4 105 & 5 107 that are exposed 106 and used 108 by the N-CoV virus for cellular invasion and release of the single stranded RNA viral vector. FIG. 7 illustrates the location of the proteins of the N-CoV virus 117. The Cysteines in the 2D structure are located as shown in red illustrated in FIG. 8.

These exposed Cysteine targets 107, 108 are available for 1-Sulfur-2-propene thiolation as described by FIG. 6c 112. Now the key is to simply enable this chemistry in the sinuses or lungs safely. This is performed by cold pressing garlic and then incubating the released alliin and alliinase in order to synthesize the 2PSP 109 in a natural oil form. 2PSP 109 can be synthesized as stated in the scientific literature (5) Gruhlke et al by aqueous incubation methods. Otherwise, 2PSP 109 can be synthesized as stated in the scientific literature using Organic Chemistry including exotic column catalytic enzymatic methods. R groups may be added to the natural or synthetic 2PSP base molecule as in FIG. 6d by leveraging the practice of the art of standard Organic Chemistry to produce the 2PSP-R structure 116. Heating 2PSP 109 can be used in the practice of the art to produce Ajoene and a number of mono, di, and tri sulfides that have been found to have cardiovascular therapeutic function in the scientific literature of Alexander et al (6). These are shown in FIG. 9.

Selenium 119 is naturally found in many foods, but is most commonly eaten in Brazil Nuts. Selenium can be cold pressed into an oil and the entire mixture also extracted into alcohol polar solution . For example 10-25 micrograms of Selenium can be used. Selenium is a heavy metal that can cause liver and kidney damage in high doses. Typically the safe daily oral consumption of Selenium is ˜55 microgram which is the approximate concentration in an average Brazil nut. Higher oral doses of 200 micrograms is acceptable during infection. Here the Selenium atom 119 replaces the Sulfur atom in the exposed Cysteine of a protein during a protein translation error whereby the translating tRNA protein 120 accidentally injects 122 a Selenocysteine 121 for a Cysteine amino acid into the 2D translated system structure (FIG. 10a & b). There are a number of various mechanisms in the literature that differ from ion substitution of the Sulfur atom (10a) to the accidental transferase injecting Selenocysteine 121 into the Cysteine position due to a wobble in the proteins involved in the enzymatic construction of the antigenic vector (i.e. a coronavirus such as N-CoV) that leads to a slight spatial recognition problem with the amino acid that translates to a modified protein 2D sequence 123 which can not fold into the proper 3D functional protein.

2PSP 109, Selenocysteine 121 and/or ionized Selenium 119 can enter in to lung cells because they are known to be permeable to the outer membrane of the cells and to the Rough Endoplasmic Reticulum (ER) of the cell where the SARS or N-CoV viral proteins are manufactured after cellular invasion by SARS or N-CoV. Here 2PSP 109 and Selenocysteine 121 can provide a dual inhibition of the SARS, N-CoV or similar viruses from properly folding into a functional 3D structure that will assemble the N-CoV virus capsule thus disabling the ability of the virus to properly assemble and replicate. Disabling the N-CoV in the ER allows the native human immune system to activate and generate antibodies for the malformed viral proteins. The malformed viral proteins are then removed by the immune system. It is absolutely critical to treat the human with a vapor in order to coat the sinuses, throat, bronchial, and lung surfaces against SARS, N-CoV, etc. because these viruses are airborne and typically infect patients through the lungs. Autopsies have shown that the most damage for SARS, N-CoV, and similar viruses have done major damage to the lungs during infection (3). Leveraging the said emulsion 200-205 with aromatic capability to shield the lungs is the practice of the art of shielding the sinuses, throat, bronchial, and lung surfaces from viral infection. Similarly the same technology can be used for single celled organisms that cause respiratory viral infections like bacteria, mold, mildew, yeast, etc.

The rapid damage to the lungs can occur while a patient is being treated by Hydroxychloroquine, Azithromycin, and traditional oral or intravenous antivirals and antibacterial. The autopsy data from Wuhan (3)(7) proved this. The 2PSP 109, 2PSP-R 116, and 2PSP/2PSP-R formulas 200, 201, 202 with elemental Selenium and/or natural Selenocysteine can be used to firewall shield the lungs from a SARS or N-CoV viral attack. In the event that the patient presents with the virus and an infection in the lungs the practice of the art is to add Glucose to the aromatic vapor for the purpose of providing the inflamed and damaged cells and energy source to repair themselves. Cysteine coding genetic take out applied to the Spike protein (2 Madu et al) conserved regions required for Angiotensin II receptor binding and cellular invasion is inhibited in mice studies of SARS (2). The constant attack by the virus can result in inflammatory conditions that the energy source glucose can resolve by allowing the native lung tissue to heal using normal in vivo cellular repair locally as an energy source. A typical aerosol or vapor solution may consist of ˜1% glucose for this purpose 200-205. A secondary condition involves the scenario where some of the surface tissues of the body are exposed like in the sinus and lung mucus membranes of the Bronchi or Alveoli. Here the 2PSP 109, 2PSP-R 116, and the respective combined Selenocysteine 121 formulas 200-205 are permeable and will easily be up taken by the infected cells and they will be absorbed into the cytoplasm where the Rough Endoplasmic Reticulum is used by the SARS 99, N-CoV 100, etc. virus to translate the mRNA of the viral genome into specific proteins required for viral assembly. Here Selenium 119 in the form of Selenocysteine 121 is incorporated in the viral protein sequences 122 keeping them from properly folding into a functional 3D geometry that must produce a conserved Cysteine bridge motif in the 3D protein structure of the Spike (2) or Envelope (1) of a coronavirus. Similarly, 2PSP 109 or 2PSP-R 116 are also able to establish a thiosulphate bond 114, 115 with the conserved Cysteines of both the spike (2) and the envelope (1) during translation from mRNA into a protein that is then structurally modified and cannot fold into the proper 3D Geometry that is able to establish a stable equilibrium with a ΔG=0. The enthalpy of the protein folding mechanism is distorted as well due to these structural changes. The practice of the art of folding proteins has been well established in the Scientific literature affiliated with the Physical Chemistry Thermodynamics of protein folding to be enthalpy specific with respect to in vivo cellular solutions. These environmental conditions for the modified Spike or Envelope easily distort the Geometry of the protein and in some cases inhibit an equilibrium condition favorable of a functional Spike or Envelope 3D protein geometry. In some cases ΔG=0 is not accomplished. This inhibits the capacity of the SARS 99, N-CoV 100, or related virus to replicate and lyse infected cells. The immune system can then leverage white blood cells and phagocytes to engulf and remove the foreign viral proteins that are not native to the human mucus membrane cell in the eyes, ears, nose, throat, sinuses, or lung. The immune response mechanisms thereof are well documented in the Scientific literature for such malformed viral antigens.

### BRIEF DESCRIPTION OF THE DRAWINGS

FIG. 1 is an illustration of the six colloidal (1-6) suspensions that support the inability of Allicin, or other molecules within each colloidal suspension from easily reacting with itself or another ingredient in the colloidal suspension. Propylene Glycol and Glycerol are used as FDA approved food safe suspension agents.

FIG. 2a is an illustration of a probability calculation for the permutation of 2D sequence for the Spike protein of the N-Cov virus as it is translated in the Endoplasmic Reticulum of a eukaryotic cell relative to its ability to fold into a proper 3D functional protein in the cells Golgi Body thus reaching an equilibrium state.

FIG. 2b is an illustration of a probability calculation for the permutation of 2D sequence for the Envelope protein of the N-Cov virus as it is translated in the Endoplasmic Reticulum of a eukaryotic cell relative to its ability to fold into a proper 3D functional protein in the cells Golgi Body thus reaching an equilibrium state.

FIG. 2c is an illustration of a probability calculation for the permutation of 2D sequence for the Surface Glycoprotein protein of the N-Cov virus as it is translated in the Endoplasmic Reticulum of a eukaryotic cell relative to its ability to fold into a proper 3D functional protein in the cells Golgi Body thus reaching an equilibrium state.

FIG. 3 is a summary of the shown probability of the 2D amino acids of the Spike, Envelope, and Surface Glycoproteins relative to the 2D amino acid sequence translated in the Endoplasmic Reticulum of a eukaryotic cell supporting the proper 3D folded equilibrium state of the Golgi body of that cell to a functional equilibrium state based on the protein sequence itself.

FIG. 4 is an illustration of the region in yellow highlight that contains conserved Cysteine amino acids that are known to form an essential disulfide bridge in the N-Cov Envelope protein.

FIG. 5 is an illustration of the region in yellow highlight that contains conserved Cysteine amino acids that are known to form an essential disulfide bridge in the N-Cov Spike Glycoprotein.

FIG. 6 is a molecular illustration of 2-Propene-1-Sulfonic acid 5-2-propenyl ester (2PSP) which is commonly known as Allicin.

FIG. 6a is a molecular illustration of 1-Sulfonated 3-Propene.

FIG. 6b is a molecular illustration of Cysteine.

FIG. 6c is a chemical reaction illustrating how Cysteine can bind to an exposed Sulfur atom of 1-Sulfonate 2-Propene.

FIG. 6d is a molecular illustration of how Flanking R groups can be added to (2PSP) for the ability to modulate the molecular interaction of the leading Sulfur atom relative to Thiol attack of the Cysteine sulfur in the FIG. 6b reaction in vitro or in vivo.

FIG. 6e is a chemical reaction illustrating how an R-group modified 2PSP moiety can attack a Cysteine amino acid Sulfur side chain in vitro or in vivo.

FIG. 7 is a simple illustration of the structure of the N-Cov SARS like virus.

FIG. 8 is a simple illustration of the N-Cov SARS like virus targets of the therapeutics discussed herein.

FIG. 9 is an illustration of the organic molecular reaction of 2PSP with itself to produce the molecule Ajoene.

FIG. 10a illustrates the differences in molecular structure between Cysteine and Selenocysteine. It depicts how Selenium can replace the Sulfur atom of Cysteine to produce Selenocysteine.

FIG. 10b is an illustration of how the protein translation machinery of the Endoplasmic Reticulum can accidentally insert a Selenocysteine amino acid in the position of the Cysteine amino acid to produce a mutation in the 2D amino acid of any translated protein in a cell. Emphasis on N-Cov is cited here.

FIG. 11a is a molecular illustration of Ethanol with emphasis on the OH polar solvent moiety.

FIG. 11b is a molecular illustration of Propylene Glycol with emphasis on the two OH polar solvent moieties.

FIG. 12 is a molecular illustration of Glycerol with emphasis on the three OH polar solvent moieties.

FIG. 13 is an illustration of a commonly used ceramic cartridge for gently heating natural plant based food that can immediately be delivered to the surface of the lungs without digestive or heating degradation.

FIG. 14a and FIG. 14b are illustrations of how the stop codon for protein translation UGA injects a Selenocysteine and continues to translate protein 2D sequences in the Endoplasmic Reticulum when higher concentrations of Selenocysteine are available to the cell.

FIG. 15 is an illustration of the pertinent Biochemical Pathways tied to the N Cov SAR like virus and the ability to interfere with the replication of the N-Cov virus within a Eukaryotic cell such as a lung cell with a known ACE2 receptor on its surface.

FIG. 16a is an illustration of how the Angiotensin II type I receptor and the ACE2 receptor are cojoined in the Golgi body of a Eukaryotic cell as a dimer that is excreted to the surface of the cell.

FIG. 16b is an illustration of how Losartan can bind this dimer in FIG. 16a when the dimer has reached the surface of the Eukaryotic cell. For example this has been published to occur in Human Lung Cells.

FIG. 17 is an illustration of the use of an FDA certified medical device that can take Human Blood Pressure readings. This particular example illustrates how a Losartan vapor formula 1 can reduce a hypertension 2 state to a hypertension 1 state. It also illustrates how an vapor formula 2 made up of the 2PSP vapor suspended in Propylene Glycol & Glycerol can normalize blood pressure that is in a Hypertension 1 state.

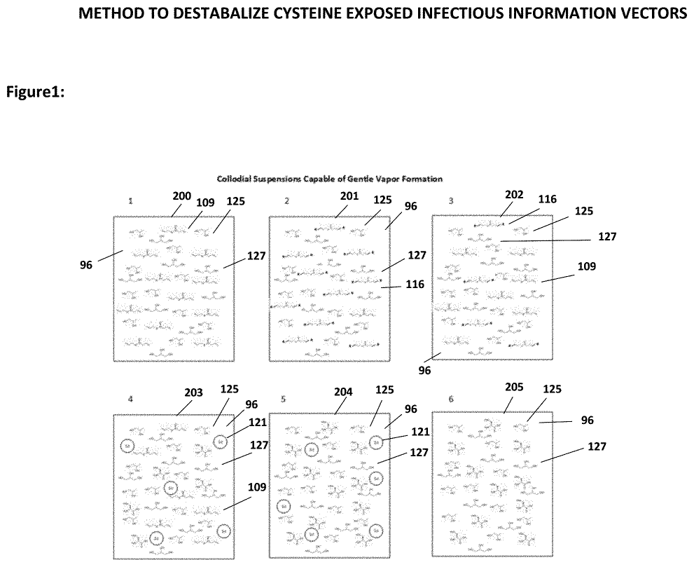

### DETAILED DESCRIPTION

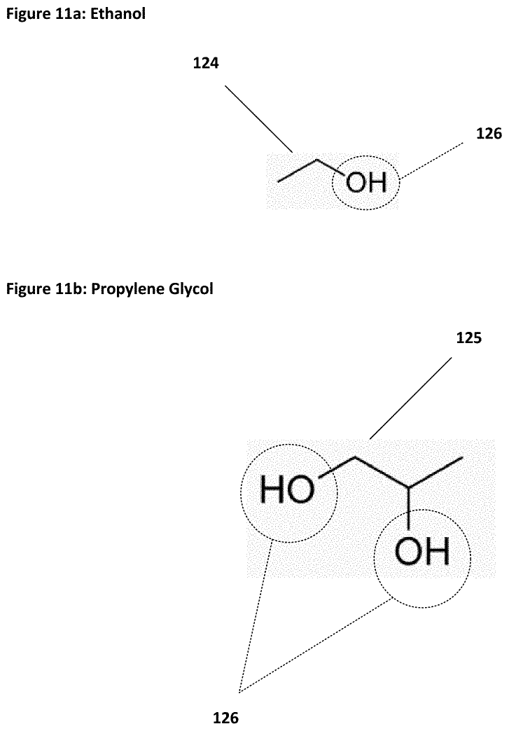

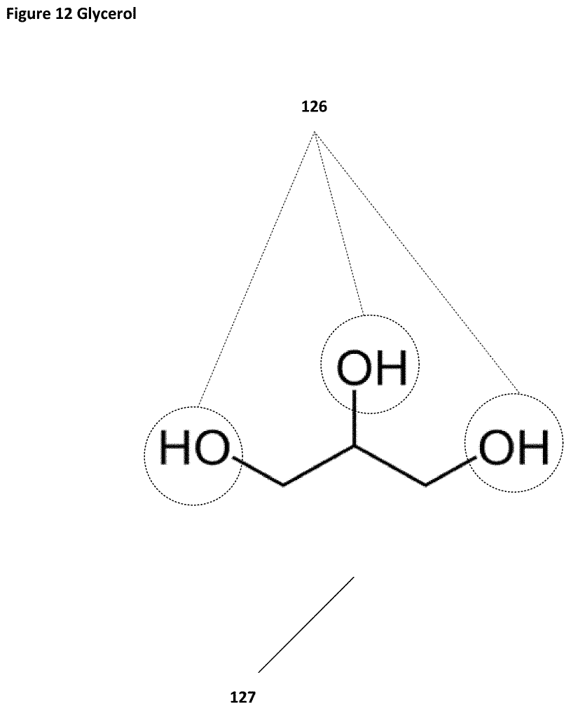

2PSP 109 is created by pressing organic native garlic thus releasing the alliin 98 and alliinase 97 from the stored vesicles within the garlic cloves. The skin of the garlic clove is removed. The resulting well known practice of the art involving solubilization in 20-40% ethanol (FIG. 11a) is then modified herein by solubilization in propylene glycol (PG) (FIG. 11b) instead of water & ethanol solution. The solubility of PG 125 relative to ethanol 124 is greater for 2PSP because 2 OH groups 126 are more polar relative to the 2 carbon atoms versus only one OH group in ethanol. PG 125 is not oxidized to form toxic aldols that result in headaches due to brain chemistry destabilization in mammals. Propylene glycol 125 is a known sanitization molecule because of the two alcohol groups 126 on the molecule, which are more effective than ethanol 124. Propylene glycol 125 is a known safe food processing agent that is allowed for oral consumption by the Food and Drug Administration (FDA) in the USA. The polarity of the PG with its two OH groups 126 allows for the greater solubility of alliin and alliinase than in water or 20-40% ethanol 124 & water 96 alone. PG 125 allows for the synthesis of 2PSP 109 also known as allicin 109. The polarity of the PG allows for the product of the synthesis of 2PSP 109 to become extracted and soluble in the PG 125 over a period of 3-5 hours at standard temperature and pressure. Upon completion of incubation of the garlic plant extracts to produce 2PSP 109 the extraction of it into the PG aqueous base solution is instantaneous as it is produced. For instance 50 ml of PG 125 may be used for the incubation & extraction process herein described for the first time. In addition glycerol shown here in FIG. 12 127, has 3 times the OH groups 126 that ethanol has and one more than PG. Glycerol has 3× the solubility of ethanol relative to 2PSP 109 because of this. Glycerol is a safe food processing agent as defined by the FDA. Glycerol is used in conjunction with PG 125 to solubilize the 2PSP 109 thiolester during incubation. The 3 OH groups 126 of glycerol 127 are also more polar than that of ethanol 124. For instance 50 ml of glycerol 127 is added to the 50 ml of PG 125 for a total of 100 ml of aqueous solution. For example 271 grams of organic garlic may be pressed in such a way that 116 grams of allicin 109 can be produced on average based on known approximate enzymatic output of the 2PSP 109 synthesis process from a garlic clove. Upon incubation and filtration using meshed 3M nylon and then 0.25 micron Whatman filter paper and a side arm Erlenmeyer brought under vacuum using a vacuum pump the supernatant is a solution containing approximately 2.7 mg/ml 2PSP 109 suspended in PG (50 ml) 125 and glycerol (50 ml) 127. The practice of the art may vary, but ours is a novel method described here relative to the preparation because glycols are used as safe alcohols. Traditionally, the published practice of the art uses ethanol because ethanol is a food processing agent that is consumable per the FDA. The problem with ethanol is that ethanol is not only regulated by the alcohol, tobacco, and firearm (ATF) division of the US Government but also must be processed by the liver of a mammalian organism like a human. Ethanol 124 is a known human toxin that is oxidized by the liver to produce ethyl aldol which can have adverse effects. In addition the vapor pressure from ethanol-based formulas is good for aerosols, but is bad for use in over the counter vapor cartridges produced by manufacturers. Ethanol 124 aqueous solutions do not slow the Brownian motion of the 2PSP 109 from colliding with itself. As a result the 2PSP 109 will collide and react with itself at room temperature or when heated. Furthermore the CRC Handbook and the Merck index have 2PSP 109 as having only ˜20% solubility in 40% aqueous ethanol 124 solution. Heating itself is well published and known to destabilize the 2PSP 109 as previously discussed in the background of this embodiment. The viscosity of the PG 125 and glycerol 127 formula 200 is such that the 2PSP 109 molecule becomes colloidally suspended in the aqueous solution with reduced Brownian motion that results in less 2PSP 109 collisions. This base formula can then be frozen at −10° F. for long periods of time. For example this base formula can then be further diluted by 0.34 ml of PG to 0.66 ml of the base formula to produce a 0.3 mg 2PSP to 1 ml volume solution. The 1 ml solution can then be mixed and added to a 1 ml vapor cartridge manufactured by any vendor. For the purpose of discussion here we will discuss the use of CCELL vapor cartridges 128 manufacture by Smoore and Jupiter research. All rights for CCELL are owned by Smoore and Jupiter Research, and we do not make any claims relative to their said invention but use them as one of many types of heating technologies available off the shelf for portable vapor production. The vapor cartridges contain the ceramic heating elements 129 that produce the proper temperature for gentle heat of vaporization of various PG 125 and glycerol 127 formulas 200-205 of specific viscosities. As previously published in the literature the porosity of the ceramic heating element wicks requires a specific viscosity of the solution. Solutions that are too aqueous and not viscous enough will not properly flow across the ceramic heating elements 129 of these vapor cartridges 128. In order to determine the proper viscosity of the mixture for a given vape cartridge such as a CCELL 128 a viscosity and thermal study was performed. The thermal study was performed by using a Smoore Vaporesso revenger II programmable power source that is capable of delivering direct current of 3 volts (DC) power across the 1.94 Ohms ceramic heating element 129 of a 1 ml CCELL cartridge 128. Trial & error was used to modify the viscosity formulation as a practice of the art to come into the range of the CCELL cartridge 128 because the manufacturing specs of the ceramic heating cartridge are not available from the manufacturer. All of the ceramic cartridge specs are proprietary to the manufacturer Smoore.

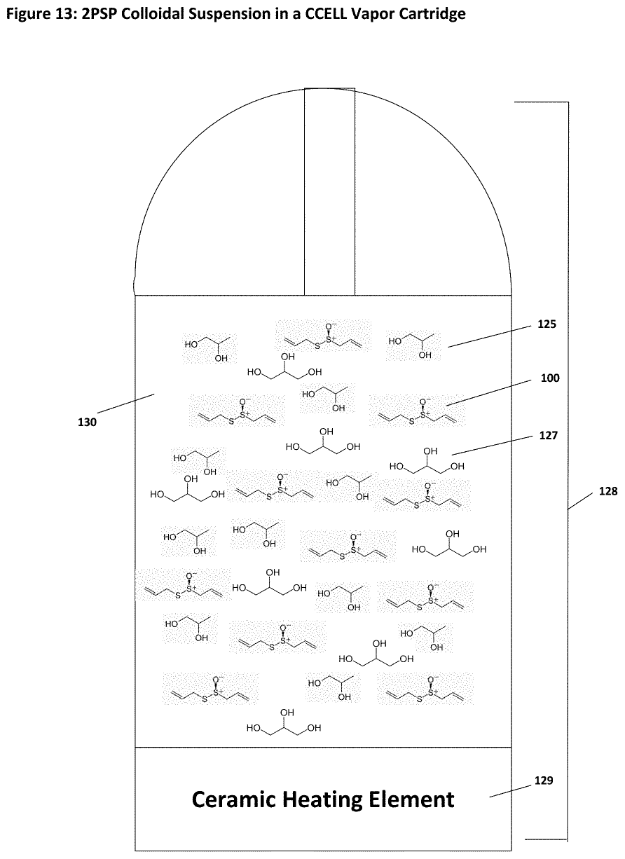

The Vaporesso revenger 2 has an on-board micro-computer capable of programmatically regulating the DC current, the power, and thus the thermal heating that is acceptable by the ceramic heating element for the 2PSP 109 formulation 200. This is how it was determined that the 0.30 mg/ml suspension of 2PSP 109 in PG 125 and glycerol 127 would be usable for a CCELL cartridge 200. We used a CCELL cartridge to formulate our solution for practical use in the CCELL, the defined practice of the art described herein can be used to produce a 2PSP 109 formula in PG 125 and glycerol 127 that can be used with other ceramic or quartz heating elements from other manufacturers. The choice of using such ceramic technology is because metal heating elements are not easily controlled within the heating ranges required to gently heat a viscous thiolester solution of this type in such a way to avoid thermal degradation. 2PSP 109 and 2PSP-R 116 are easily degraded by thermal heating. 2PSP 109 and its R group derivatives 116 are easily degraded even at room temperature in non-viscous aqueous solutions over time because of molecular collisions that can occur. The described practice of the art formulation example here allows us to suspend the 2PSP 109 molecules in such a way that they not only become stable and colloidally suspended in solution 200-203, but are also stable and kept away from the ceramic heating element so that destabilization of 2PSP 109 in solution does not occur. The formula is designed to only introduce 2PSP 109 to the heating element 129 at time of vaporization due to inhalation because the colloidal suspension of the 2PSP 109 molecules in the 1 ml vial of the CCELL vapor cartridge used as an example here. Other vapor cartridges may have larger volumes or different pores in the ceramic heating elements may require a viscosity adjustment by varying the PG 125 or glycerol 127 concentration. This will allow for using those vapor producing devices as well. The practice of the art may require minor changes to the viscosity that inhibit 2PSP 109 from reacting with itself. FIG. 13 illustrates the separation of the molecules in the suspension for this purpose 130.

These viscous colloidal emulsions 200-205 require special heating that can be performed using ceramic resistors 129. The ceramic resistors 129 in a CCELL vapor cartridge 128 can maintain the ˜35° C. temperatures allowing the emulsion 200-205 to go into vapor and reach all of the surfaces of the lungs, bronchial, throat, and sinuses. Variable controlled voltage can be used in the electronic circuit across the ceramics to create different heating conditions that favor 2PSP 109 or 2PSP-R 116 vaporization. Some 2PSP 109 self-reaction occurs to form Ajoene on the surface of the ceramic heating element. Ajoene 118 is known to have therapeutic action on the blood and circulatory system when allowed to enter the blood. Ajoene 118 is known to thin mammalian blood and reduce blood pressure. Specifically Ajoene 118 reduces the coagulation of Von Willebrand Factors in locations of the blood were the mucosal walls of the said blood vessel, artery, capillary, etc. are inflamed. The ability to put 2PSP 109, 2PSP-R 116, and/or Ajoene 118 with high availability into the surface of the lungs allows high surface bioavailabilities of 99%+ to be achieved on the surface of the alveoli of the lungs. 2PSP 109, 2PSP-R 116, and Ajoene 118 are known to exist in oral products that enter the GI tact. These molecules can also exist in intravenous forms. The problem as previously mentioned is that 2PSP 109, 2PSP-R 116, and/or Ajoene 118 will decompose through interactions with other biological compounds existing in the GI tract or the blood. This will cause the bioavailability to the Alveoli of the lungs to be extremely low. In the case of a ˜140 nm in size coronavirus you must provide a monolayer of 2PSP 109 directly to the lungs. It is impossible to do this with oral or intravenous solutions due to the stated decomposition. Thus the described colloidal solution is used to achieve vapor nanocoating of the lungs for antigen shielding in an immediate way by means of inhalation into the lungs. In the case of a coronavirus the ability to monocoat the lungs and Alveoli of the lungs allows exposure of the 2PSP 109 or 2PSP-R 116 to the digitally coded antigen.

For example in the case of N-CoV 100 the viral spike 107 protrudes from the viral vector 117 as an antigen. The viral spike 107 is coated with glucose molecules that shield the spike 107. These are used as a glycolytic energy source that is used by the host cell for energy in the absence of Oxygen in order to power the glycolysis required to power the digital DNA/RNA viral replication of N-CoV. The glycosylated spike 107 requires the use of a small molecule that is capable of traversing the glycosylated outer portion of the spike 107 and reaching the exposed Cysteine bridge 107b of the spike 107 identified in N-CoV that exists in all functional coronaviruses as published by Madu et al (2). Our identification of the continued conserved Cysteines 107b in N-CoV 100 on Feb, 2, 2020 was the first ever. This revelation led to the vapor formulation design to attack this spike 107 Cysteine bridge 107b on the surface of the lungs. However, 2PSP 109 and 2PSP-R 116 are permeable to the semipermeable membranes of the human cells on the surface of the lungs. Unlike a coronavirus such as N-CoV 109 the spike 107 is not directly permeable to the membrane of the cells because its size and physical properties are simply too large to permeate the semipermeable membrane. Instead the spike 107 of N-CoV 100 must bind to the ACE2 receptor of an intramembrane protein on that cell in order to be engulphed. This has been published and is well known in the scientific literature to date. Because 2PSP 109 or 2PSP-R 116 can enter the cell by traversing the semipermeable membrane the 2PSP 109 and/or 2PSP-R 116 molecules can proceed through Brownian motion of the aqueous cellular solution into the Rough Endoplasmic Reticulum (RER) of the cell where the digital codes for N-CoV 100 hijack the host cell and reverse translate into a protein encoding for the N-CoV spike 107. As the 2D translation occurs in the RER the 2PSP or 2PSP-R binds the 2D added Cysteine thus disabling its ability to later fold into a functional 3D protein 107 in the Golgi Body. Upon completion of the translation of the entire spike protein 107 into a 2D amino acid polymer the protein will migrate to the Golgi Body of the cell. At this point the protein will attempt to fold 3 dimensionally. The 2-dimensional protein sequence will fail to properly fold into a proper 3D structure that has proper spike 107 functionality for N-CoV 100. This knock out of the Cysteine amino acid side arm sulfur atom 111 in the spike 107b disables its ability to allow ACE2 binding during infection because the essential Cysteine bridge is not formed in the spike protein 107. The result of that creates an immune response by the well-known and scientifically published literature on the human immune system and how antigens are removed through immunity.

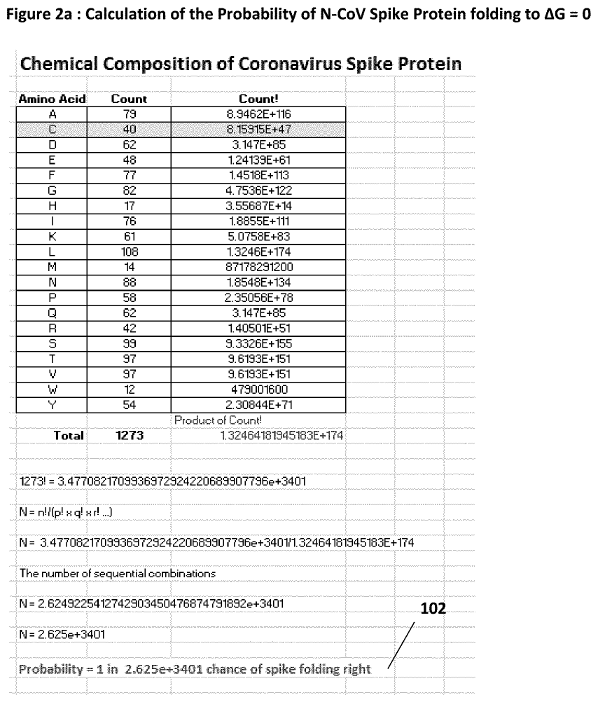

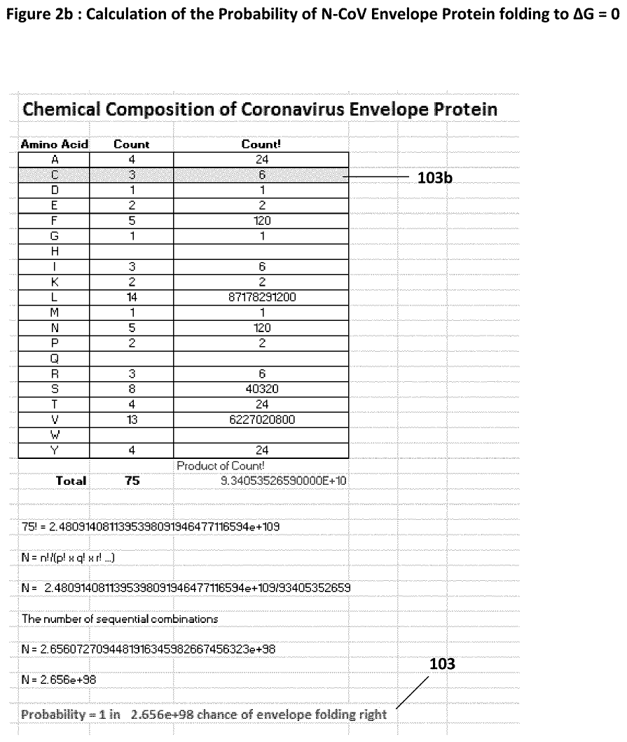

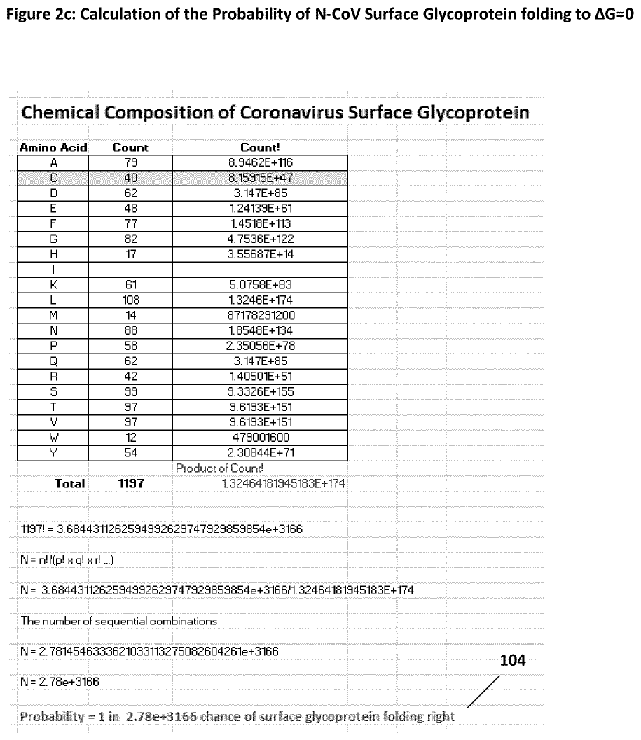

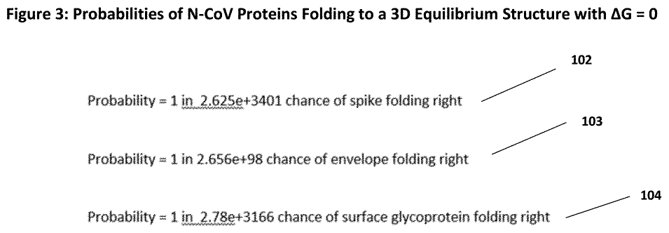

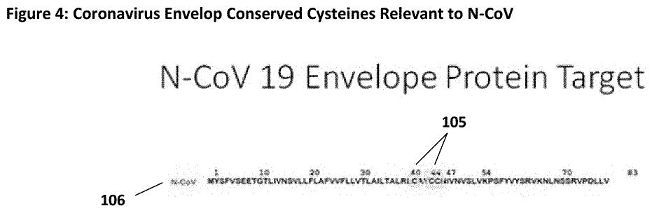

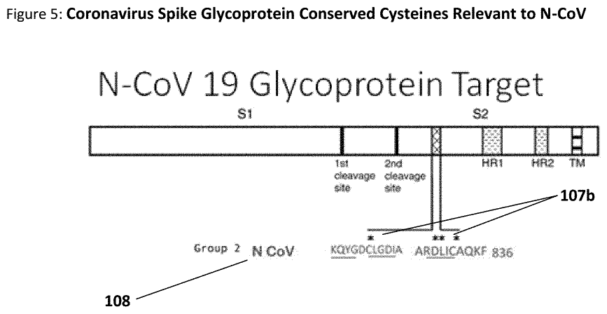

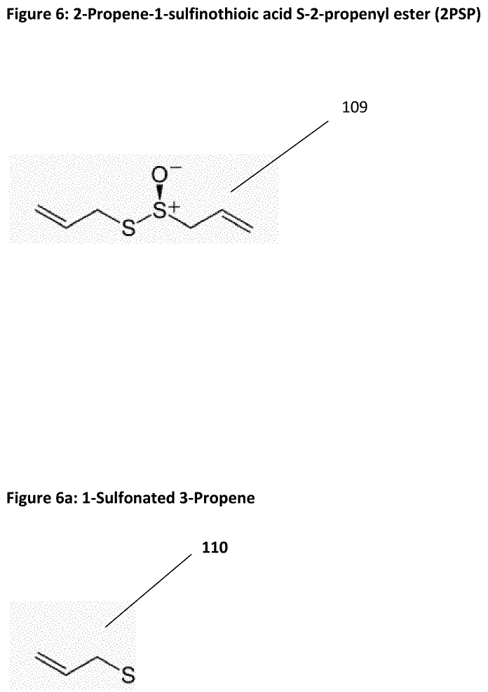

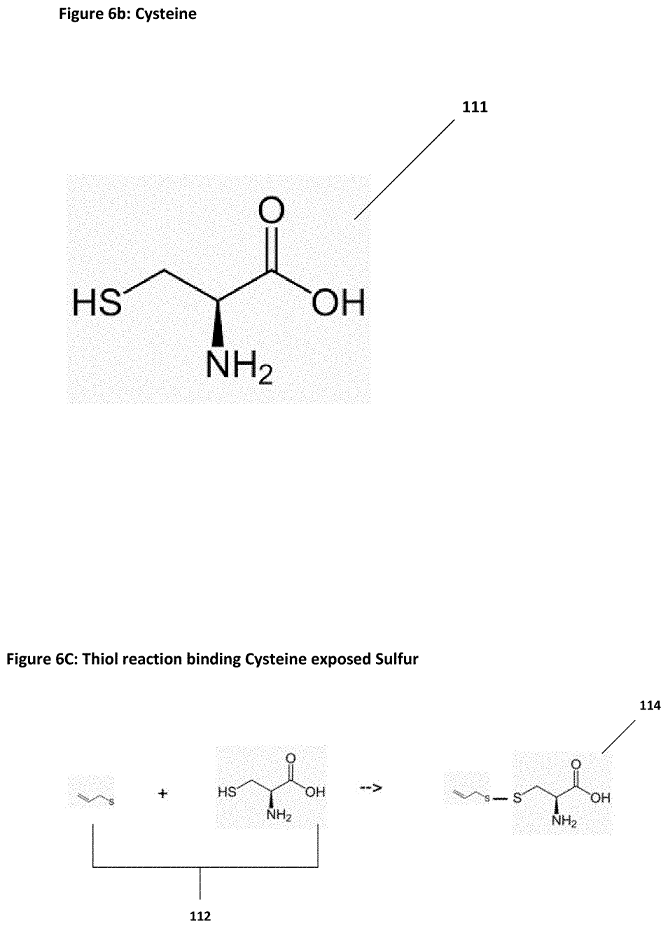

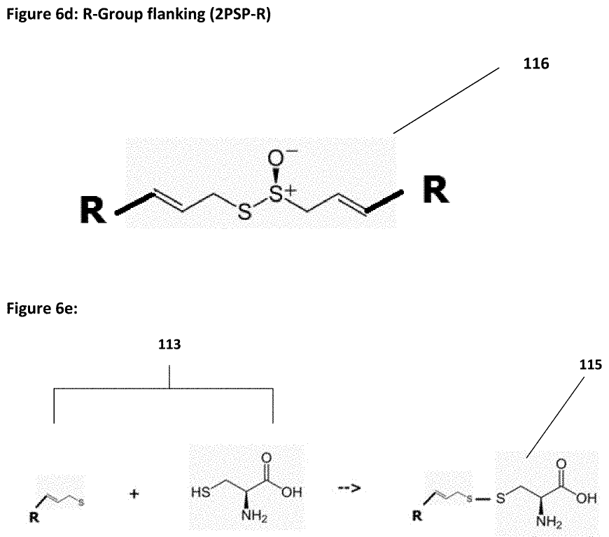

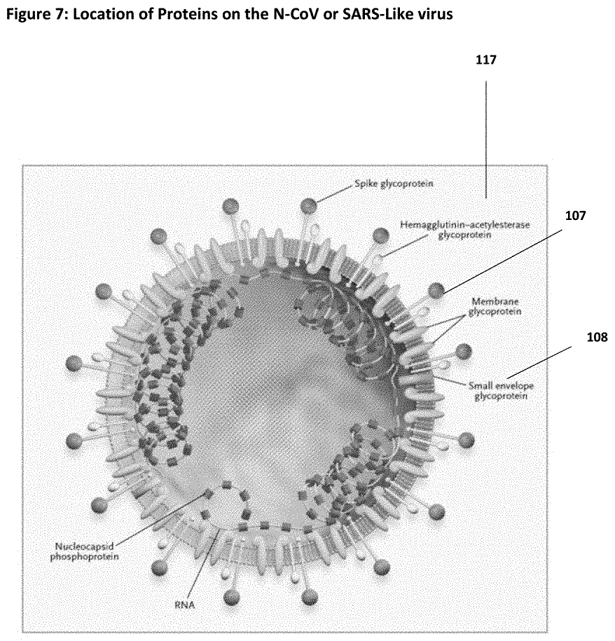

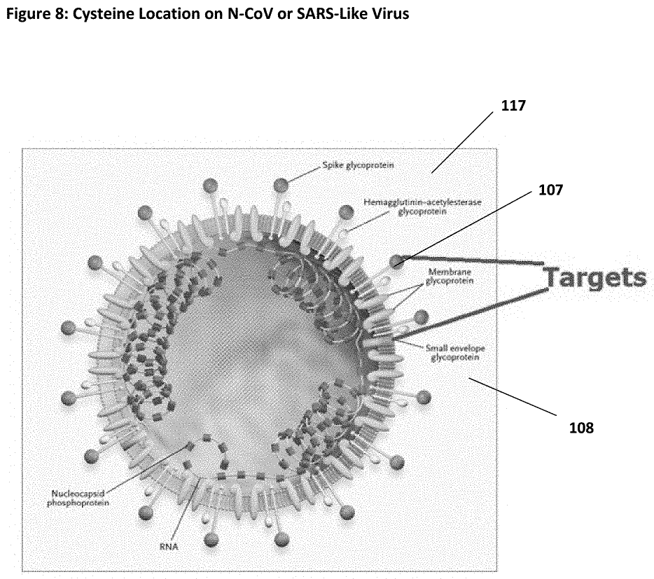

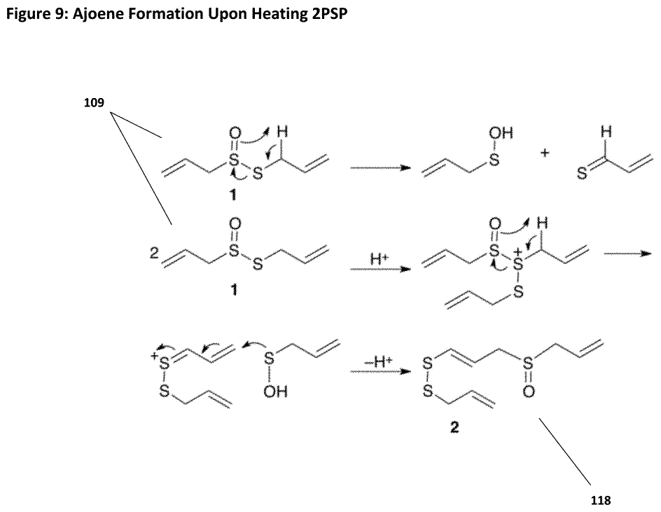

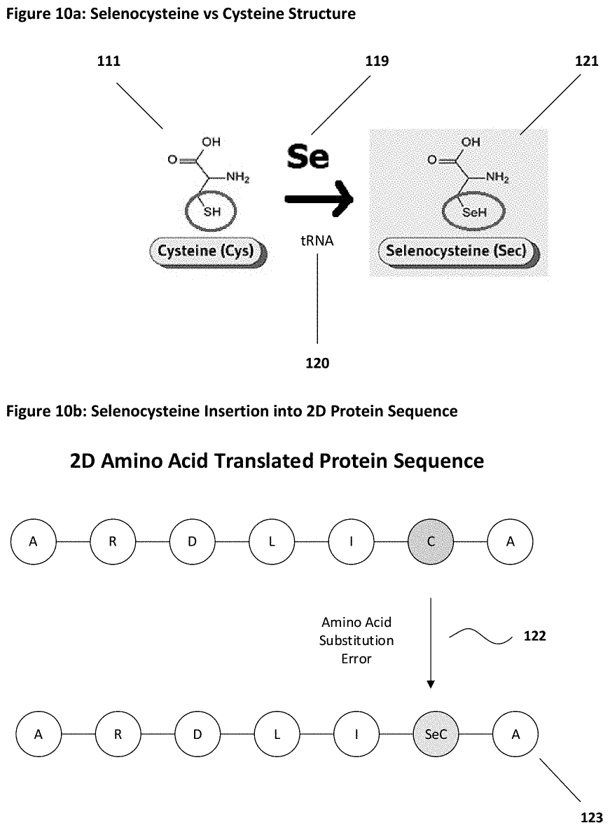

A secondary mechanism is even more probable based on the probabilities 102, 103, 104 computed and shown previously in FIG. 2b for the N-CoV envelope protein. Here there are only 3 Cysteines in the envelope of any coronavirus 103b. In the case of N-CoV 100 the translation knock out of only one of the two essential Cysteines 103b is required in order to keep the envelope protein of the N-CoV 100 from properly folding into the proper 3D protein geometry. As a result a malformed envelope as discussed in the gene knock out wild type work published by Lopez et al (1) is disabled during translation with higher specificity than the same mechanism for the spike protein. In both cases the kinetics of viral digital code reverse translation is greatly slowed in such a way as to allow the mammalian immune system to overcome and clean up the viral infection. Normally, the viral replication process would proceed to increase at an exponential rate. Here that is deterred at the 2-dimensional protein translation step in protein synthesis for the said viral foreign viral envelope protein 108 that is digitally coded for by the codes of the N-CoV 100 itself.

Consumption of 2PSP 109, PG 125, a Glycerol 127 are safe as described in the regulatory food processing literature provided publicly by the FDA work to determine the safety of R-group modifications of 2PSP 109 to produce 2PSP-R molecules 116 must be performed using the traditional animal safety testing methods, and clinical methods used in the practice of the art relative to bringing forth any sort of FDA approved therapeutic in the USA. This is a requirement by the US Government for product labeling. It however, is not a requirement for the sale and consumption of processed food items using the said safe molecules on the FDA list without labeling beyond food processing.

The one problem with 2PSP 109 and 2PSP-R 116 are the shelf life storage of the molecules in the colloidal suspension. Our thermogenic study indicated that a 0.3 mg/ml solution in a glass vapor cartridge such as a CCELL vapor cartridge 128 would have an approximate 30-day shelf life with regular limited daily usage 200-205. The colloidal suspension 200-205 allows for the shelf life because the viscosity of the mixture is such that heating does not cause degradation of the 2PSP 109 or 2PSP-R 116 molecules. There were no adverse human affects from the inhalation of this 2PSP 109 formula on humans with a limited study over the course of one year. Additionally our limited study was conducted on a single human with diabetes and a known Eosinophilic respiratory disease. No adverse effects from this limited study occurred. Further studies need to be performed on animals and humans in order to validate inhalation safety. However, the FDA has already approved these molecules for food safety based on published research. So, the expectation of an inhalation safety issue for normal inhalation is minimal. In addition in vitro studies validated that 2PSP 109 can be used as a vapor antimicrobial that inhibits bacterial infection (4).

A more advanced formula 203, 204 was created using Selenocysteine 121 because the 2PSP 109 are unstable molecules, and the fact that they have limited capability during digitally encoded viral infection. Selenocysteine 121 can be added to the formula 203, 204 such that for instance 25 micrograms per milliliter exists. This will result in a sub-nanogram vapor aliquot released by the cartridge into the lungs with each inhalation from a vapor cartridge 128. Selenocysteine 121 is a natural amino acid that is not one of the 21 essential amino acids for life. Selenocysteine 121 is the 22^{nd} amino acid. Selenocysteine 121 is encoded for by the protein translation stop codon. Typically Selenocysteine 121 is not present in high concentrations in a mammalian organism. Humans can live with low concentrations of Selenocysteine 121 just fine. The oral consumption of Selenocysteine 121 is allowed by the FDA as a vitamin supplement. The oral and intravenous consumption of Selenocysteine 121 can cause a heavy metal toxicity condition that is well published and mentioned previously herein the background of the embodiment. Selenocysteine 121 like 2PSP 109 is highly reactive with tissues of the body because like all amino acids they are permeable to the semipermeable membranes of the eukaryotic cells of the mammalian tissues of humans.

Therefore the use of Selenocysteine 121 in a vapor formula allows for specific and immediate targeting of the human lungs. Selenocysteine 121 is then up taken by the surface cells of the lungs and is allowed to proceed to the RER where digital translation of the viral codes of N-CoV 100 occur. Here you have 2 effects that come into play. The first effect is the ability of Selenocysteine 121 to inject in the Cysteine position when the tRNA transferase 122 codes for Cysteine because a wobble in the codon translation protein(s) can mistakenly inject a Selenocysteine 121 amino acid into the 2D sequence of the spike 107, 107b or envelope 123. This will disable the ability of that 2D protein sequence from folding into the proper functional 3D protein structure. This occurs specifically when the Sulfur atom is replaced by a Selenium atom from Selenocysteine 121 , which will not allow that Sulfur atom to form a bridge with another Sulfur atom from a corresponding Cysteine amino acid 111 in the juxtaposition of a 3-Dimensional viral (etc. digitally coded antigen) protein. It will then allow the viral protein replication assembly kinetics to become dysfunctional in such a way that the mammalian immune response will cleanup all of the existing viral protein debris as normal. This is well published in the scientific literature relative to viral immunology.

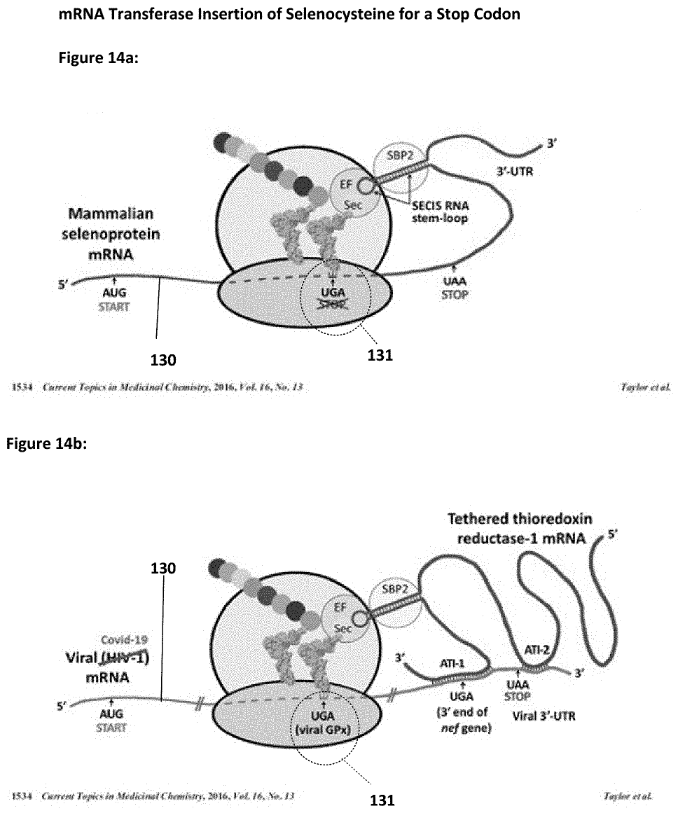

The second digital exploit that becomes enabled by providing Selenocysteine 121 to the surface cells of the lungs using vapor is the fact that the stop codon 131 in protein translation in the presence of a higher concentration of Selenocysteine 121 will in fact not stop translation. Instead the translating protein machinery will instead inject a Selenocysteine amino acid 121 and then continue translating the mRNA sequence 130 injecting other amino acids. A higher concentration of Selenocysteine 121 during protein translation can malform the spike 107 and envelope proteins 108 of a coronavirus like N-CoV (FIG. 14a-b) 100 as described by Taylor et al relative to Ebola or HIV-1 per Taylor et al (8). Here we have a different condition that also leads to a 3D protein folding anomaly for the spike 107 and the envelope 108 of N-CoV 100. When this happens the 3D geometrical structure produced by the protein folding process in the Golgi body is dysfunctional and inhibits proper viral capsid formation 117. The result is massive slowing of the coronavirus replication mechanism rate. When the replication rate kinetics are massively slowed the well scientifically published mammalian immune response by the organism is allowed to come in and clean up the protein debris of the malformed virus. This of course is only fully enabled at the surface of the lungs by using a vapor derived from a colloidal suspension 200-205 as defined here with or without 2PSP 109 as described herein.

The concentrations of Selenocysteine 121 can be brought up in a mammalian organism like a human over time by orally consuming Selenocysteine 121 thereby making the rare amino acid available for translation Biochemistry. However, this does not immediately increase the local Selenocysteine 121 concentration at the surface of the lungs like the herein described mechanism does. The viral replication rates are exponential and time is of the essence when the digital codes of a coronavirus 100 enter a host cell. It is essential to immediately bring the Selenocysteine 121 to the infected cells using the vapor mechanism in order to slow the exponential viral replication rate kinetics immediately. This is not even possible with intravenous injections of Selenocysteine 121 due to the permeability of the amino acid across the semipermeable cell membranes of a mammalian cell that result in uptake of tissues that are not the primary infection tissue of an environmental virus like a coronavirus 117, 100.

The practicality of this technology is such that it can be made available to the masses in such a way that large portions of the populations can gain access to the vapor formulas 200-205 through just in time manufacturing using the same conventional packaging methods used by the tobacco and cannabis industry infrastructure in place today. The colloidal suspension formula production does not have any special refrigeration requirements that makes it more complex relative to distribution to the masses. The effects of refrigeration or freezing is such that the viscosity of the solution decreases and allows long term storage of the P2SP 109 without degradation.

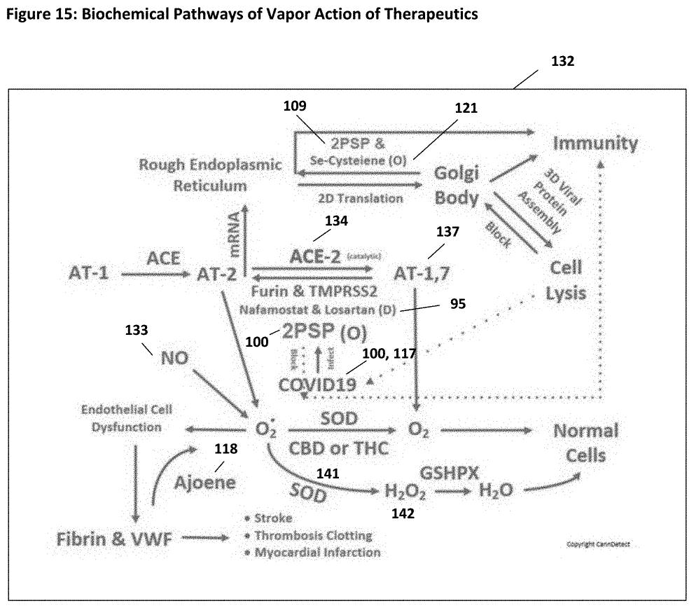

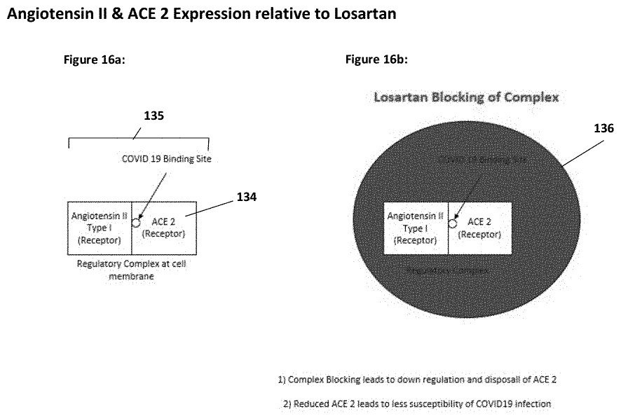

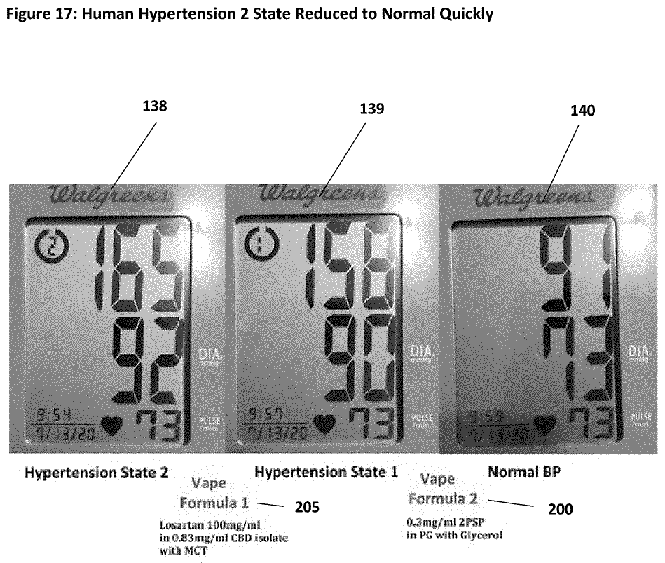

The Biochemistry 132 of using the vapor formulas discussed herein the embodiment is easily described using the typical Biochemical pathway of the molecules used in vapor form 200-205. Here we studied the use of several other vapor formulas that are currently available in the USA Markets by means of prescription to show the synergistic effects as they work with the 2PSP 109 vapor formula in conjunction with Selenium from Selenocysteine 121 to produce synergistic effects (FIG. 15). LSU Medical School in Shreveport, LA has significantly studied the effects of Nitrous Oxide (NO) 133 shown in FIG. 15 as having an Oxidative radical reduction effect on N-CoV 100 patients. This is due to the known Biochemistry pathways 132 that NO 133 has relative to in vivo behavior that has been established in the Anesthesiology scientific field and validated through clinical work at LSU (9). Anesthesiologists there have been treating N-CoV 100 patients with NO for 30 mins two time a day. It is therefore shown here as they have had success using NO 133 vapor because it was readily available to them in bottled tanks. 2PSP 109 is typically not available in tanks because as we discussed it is unstable and can react with itself under pressure or heat. It is also known in the scientific literature that NO 133 therapy can reduce radical oxidation from sugars that created oxidative radicals in the blood. It is known that the N-CoV 100 (labeled COVID19 here in FIG. 15) has a spike protein 107 that must bind with the ACE-2 receptor 134. Here we used a 100 mg/ml Losartan suspension in 0.83 mg/ml CBD 141 in 1 ml of MCT Coconut pure oil 143 to produce a vapor formula that produced a white colloidal suspension in solution that was usable in a CCELL vapor cartridge 128. This mixture allowed sub microgram vapor aliquots to immediately reduce the blood pressure of a human with a Hypertension 2 state within seconds during periods of respiratory distress. The mechanism discussed in Deshotels et al (10) is that the Angiotensin Type II receptor and the ACE-2 receptor 134 not shown here are expressed through mRNA transcription at the same time and when packaged in the Golgi Body are folded and brought to the surface of the mammalian lung cell together 135. When this happens the N-CoV 100 (a.k.a. COVID19) binding domain of the ACE2 receptor 134 is not exposed, but is dimerized to the Angiotensin II receptor 135 as it is excreted towards the surface of the cell in vesicles prepared in the Golgi body of the cell FIG. 16a. Losartan 95 as a vapor is well documented to function as an Angiotensin Type II Receptor Blocker (ARB) in the scientific literature. Deshotels et al (10) show that not only does down regulation of ARB cause down regulation of ACE2 134 by Biochemical transcription relation, but also that Losartan 95 can bind the complex of ARB 135 and ACE2 134 when the dimer reaches the surface of the cell before the ACE2 receptor 134 is separated on the surface of the lung cell as shown in FIG. 16b. The rate at which this occurs when Losartan 95 is used in a vapor form is instantaneous versus the rate at which it occurs when Losartan 95 is consumed through the oral pill fashion which takes hours to be absorbed into the blood. Losartan 95 and other ARBs 136 are known to have some unusual side effects. People need to consult physicians and discuss the consumption of Losartan 95 with licensed Pharmacists. Treatment of a patient with coronavirus causing respiratory distress that also has hypertension using the 2PSP 109 vapor formula 200 discussed here will reveal a secondary blood pressure reduction by disabling the N-CoV 100 spike as shown in FIG. 17. For the purpose of documentation we show here how the catalytic reduction of ACE2 134 to AT-1,7 137 in conjunction with Ajoene 118, NO 133, and all of the well known and published oxidative reduction processes also apply to a digitally encoded virus like N-CoV 100. Here we show how a Hypertension 2 138 state is immediately reduced to a Hypertension 1 139 state of a human in terms of blood pressure using Losartan vape formula 1 205 in FIG. 17. Then we show how the blood pressure of the human is reduced to normal using the 2PSP formula 200 discussed in the embodiment herein as vape formula 2. The data was repeated many times with the same results. Every time the vapor formulas immediately reduced the patient's blood pressure to normal 140 within a few minutes because the bioavailability of the functional molecules reached the lungs instantaneously and were able to cause the effects documented in the scientific literature (10). More research needs to be performed in this area in order to collect more data relative to the safety and efficacy of these vapor formulas. We have the exact behavior as illustrated in FIG. 15 relative to N-CoV 100 (a.k.a. COVID19). The same patient had a medical prescription for CBD 141 and/or THC 142 and the traditional effects of Oxidative cellular reduction previously published in U.S. Pat. No. 6,630,507 that were validated using cyclic voltammetry which have been discussed in the scientific literature over the years where observed (13) (14). The inhibition of a coronavirus like N-CoV 100 with a 2PSP 109 formula with Selenocysteine 121 helps to deter the ACE2 134 binding as illustrated in the FIG. 15 flow diagram using the mechanisms 132 previously discussed in this embodiment. The mechanisms by which 2PSP 109 or Selenocysteine 121 hinder the rapid reverse transcription & translation of the N-CoV 100 digitally coded viral vector in the mammalian cell result in problematic 3D folding of the viral proteins thus resulting in viral replication dysfunction allowing the human body to invoke an immune response that allows cleanup of the unpackaged RNA virus in the viral capsid which leads to lysis of the cell (FIG. 15). Viruses in the class of coronavirus have ˜40,000 different flavors that exist that are well documented in GENBANK. All of these have the conserved Cysteines 105, 107b previously identified herein using GENBANK and also in the scientific literature (1) (2). These like any digitally encoded microbial or vector-based antigen are easily slowed or negated using angstrom sized molecules like 2PSP 109 or Selenocysteine 121. The problem with the large therapies targeting the spike 107 in vitro or in vivo is that they are simply too large to circumvent the glycosylated spike protein of N-CoV 100. 2PSP 109 is small enough that it can shield the mucosal tissues of the lungs by disabling the Cysteine bridge 107b in any coronavirus like N-CoV 100 that are of type SARS 99. The shielding however requires all of the novel vapor capabilities of the 2PSP 109 vapor formulas that we discussed herein. Additionally the digital code exploits require immediate bioavailability of the molecules to the surface of the mucosal cells on the target tissues of infection. This is of course impossible with the active molecules 2PSP 109, 2PSP-R 116, or Selenocysteine 121 using the traditional oral or intravenous mechanisms, because it destroys the efficacy of the Organic Chemistry used by the molecules. In order to circumvent that process the molecules are vaporized gently into a gaseous state using PG 125 and glycerol 127 that preserves them thermodynamically and allows them to quickly reach the mucosal cells of the tissues lining the surface of the sinuses, throat, bronchi, or lungs. We are using extremely small molecules in the sub-nanometer size in order to do the Biochemical work illustrated in FIG. 15 as previously discussed. One unique feature of enabling therapeutics by way of vapor 200-205 is that after inhaling the vapor the gaseous molecules can be held within the lungs allowing a greater molecular nanocoating of the surface of the respiratory tissues to occur. Holding the nose and inflating the sinuses with the exhaled vapor allows all of the tissues of the sinus cavity to become coated with the therapeutic molecules in the vapor formulas discussed herein. Typically this is not possible when an oral, intravenous, or aerosol is used to treat the sinuses. Inflamed sinuses can be inflated by exhaling from the lungs into the sinus cavity immediately thus allowing vapor-based therapeutics with the Biochemical mechanisms discussed herein to reach all of the entire surface of the sinus cavities including the ear canals with the therapeutics discussed herein when inflated this way in a highly bioavailable way that cannot be immediately performed any other way than with a vapor.

Protein translation and protein folding in vitro is a practice of the art typically used in laboratories studying protein function after a protein has been expressed in a micro-organism like bacteria, yeast, mammalian cell line, etc. There are a few different methods of purifying and attempting to crystalize the proteins so that X-ray crystallography can be performed to identify the structure of the protein. One skilled in the practice of the art knows that there are reasonable temperatures to incubate the micro-organisms they culture and also the protein isolates they are trying to crystallize. This is because the temperature range usually effects the function of the protein itself. The best observations are temperature changes that effect the rates of catalytic function of proteins that have enzymatic function. Viruses, bacteria, and other pathogenic digitally encoded entities rely on the proteins of the host organism to function at the optimal rates in order to hijack the function of the host cell. There has so far been a long discussion of how to intervene on the respiratory infection by an N-CoV 100 virus that takes advantage by hijacking the DNA & RNA polymerases of the host cell or the DNA & RNA amino acid translation and polymerization methods. These normal mechanisms are throttled to rates that are higher than normal for the targeted host cell. Additional energy is required in order to maintain those rates. In the case of N-CoV 100 the energy is in the form of sugar that the virus caries on the glycosylated spike protein 107, but also in the cytoplasm of the mammalian cells from which it hijacks after infection for the purpose of replicating. The use of P2SP 109, 2PSP-R 116, Selenium ion, or Selenocysteine 121 vapor formulas 203-204 is enhanced by changing the environment of the mammalian host cell proteins. This is performed by changing the equilibrium state of the already folded 3D proteins of the cell required to allow cellular hijack. Heating of the mammalian cells to temperatures as high as 130+° F. causes an enthalpy change related to the protein folds of the 3D structures of the enzymes involved in the polymerization of the pathogen digital codes 100 and proteins required for replication. This is typically known as heat shock for a digital code that is attempting to hijack host cell machinery at an abnormal rate in order to replicate its own digital codes. When heat is introduced to the machinery the scientific literature tells us that the enthalpy of the 3D protein structures changes, such that the 3D folded confirmation at ΔG=0 changes slightly. This happens because the thermodynamics of the 3D protein structures being hijacked follow the thermodynamic equilibrium equation ΔG=ΔH−TΔS like anything else per the scientific literature (11)(12).

When this equilibrium is shifted by changing the equilibrium state of the 3D folded proteins subjected to hijack and those that are being introduced by the digitally encoded pathogen the rates of catalytic replication function required by the digitally encoded pathogen are changed such that more translation errors occur. This allows the vapor agents discussed here to function at an even more optimal nature. Allowing the 2PSP 109, 2PSP-R 116, Selenium ions, or Selenocysteine 121 to be immediately placed at the point of infection by the digitally encoded pathogen becomes more than just being at the right place at the right time relative to optimal bioavailability, It becomes a shift in function of the cellular operating system that the digitally encoded pathogen 100 is attempting to hijack. Fortunately this allows the vapor agents to take advantage of the dysfunction introduced into the catalytic protein function of the 3D nano-machinery in such a way that the rates of viral replication are impacted greater due to unfolding of the normally folded proteins of the host cell required for pathogen replication. The further slowing or negation of the digital pathogen replication and translation of the protein sub-components allows the 2PSP 109, 2PSP-R 116, Selenium ions, or Selenocysteine 121 to then have a more profound effect on the infection rate of the pathogen and the ability of the pathogen to replicate and translate into its sub-components. That is because a heat change does not affect the equilibrium state of the molecules used in the vapor formulas herein discussed 200-205. This of course is leveraged by making the molecules available to the center of infection immediately by providing them to the place of entry of the digitally encoded pathogen to the host cell as well as allowing the discussed agents to enter the semipermeable membranes of the host cells in order to mitigate the digitally encoded pathogen at the point of cellular hijack.

1-17. (canceled)

18. A method of treating a respiratory pathogen infection in a patient, comprising:
having a liquid mixture comprising:
(a) allicin or derivative thereof;
(b) polypropylene glycol;
(c) glycerol;
aerosolizing the liquid mixture;
inhaling the aerosolized liquid mixture into the patient's respiratory tract;
contacting the liquid mixture with the pathogen in the respiratory tract;
wherein the allicin disrupts or impedes the formation of one or more disulfide bridges of a pathogen protein.

19. The method of claim 18, wherein the disruption or impediment of the disulfide bridge(s) impairs the ability of the pathogen protein to bind to an entry receptor on a host respiratory cell.

20. The method of claim 19, wherein the entry receptor is angiotensin-converting enzyme 2 (ACE2).

21. The method of claim 18, wherein the disruption or impediment of the disulfide bridge(s) causes misfolding of the pathogen protein.

22. The method of claim 21, wherein the disruption or impediment of the disulfide bridge(s) causes reduction in survival of the pathogen.

23. The method of claim 18, wherein the pathogen is a coronavirus.

24. The method of claim 23, wherein the pathogen protein is a spike or envelope protein of the coronavirus.

25. The method of claim 24, wherein the coronavirus is SARS-CoV.

26. The method of claim 18, wherein the aerosolized liquid mixture is inhaled into the patient's lungs or sinus cavities.

27. The method of claim 18, wherein the liquid mixture further comprises selenocysteine.

28. The method of claim 18, wherein the amount of allicin in the liquid mixture is about 0.27 mg/ml or about 0.3 mg/ml.

29. The method of claim 18, wherein the liquid mixture further comprises an angiotensin type II receptor blocker (ARB).

30. The method of claim 29, wherein the ARB inhibits angiotensin-converting enzyme 2 (ACE2) separation from an [ACE2]-[angiotensin-II receptor] heterodimer during transport to a cell surface.

31. The method of claim 18, wherein aerosolizing the liquid mixture comprises heating the liquid mixture.

32. The method of claim 31, wherein liquid mixture is heated to about 35° C.

33. The method of claim 31, wherein liquid mixture is held in a vaporizer cartridge comprising a heating element.

34. The method of claim 24, wherein the coronavirus has substantial homology with SARS-CoV.

35. The method of claim 20, wherein the disruption or impediment of the disulfide bridge(s) causes misfolding of the angiotensin-converting enzyme 2 (ACE2), thereby resulting in lowered blood pressure.

36. The method of claim 18, wherein the liquid mixture comprises an allicin derivative having the formula:

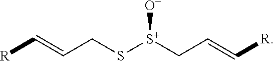

37. The method of claim 18, wherein heat shock of the human body increases the enthalpy of the proteins involved in pathogen replication such that protein folding is easier to destabilize.
